(19) United States
(12) Patent Application Publication
(10) Pub. No.: US 2013/0158434 A1
(43) Pub. Date: Jun. 20, 2013

(54) APPARATUS FOR VOICE ASSISTED MEDICAL DIAGNOSIS

(71) Applicant: DELTA ELECTRONICS, INC., Taoyuan Hsien (TW)

(72) Inventors: Jia-Lin SHEN, Taoyuan Hsien (TW); Rong-Chang LIANG, Taoyuan Hsien (TW)

(73) Assignee: DELTA ELECTRONICS, INC., Taoyuan Hsien (TW)

(21) Appl. No.: 13/693,604

(22) Filed: Dec. 4, 2012

Related U.S. Application Data
(60) Provisional application No. 61/578,091, filed on Dec. 20, 2011.

(51) Int. Cl. A61B 5/00

(52) U.S. Cl. CPC A61B 5/4803; A61B 5/7246; A61B 5/7282; A61B 5/746
USPC 600/586

(57) ABSTRACT

An apparatus for use in voice assisted medical diagnosis including a database, an input unit, a voice matching unit and a diagnosis unit. A voice model associated with an individual is stored in the database. The input unit receives a voice signal from the individual. The voice matching unit matches the voice signal with the voice model. The diagnosis unit diagnoses whether the individual suffers from one or a multiple of predetermined diseases according to a matching result from the voice matching unit. The apparatus further includes a speech recognition unit, analyzing the individual's voice response to a plurality of predetermined questions to determine one or a multiple of medical conditions of the individual. The diagnosis unit diagnoses whether the individual suffers from one or a multiple of the predetermined diseases according to the matching result and the one or multiple of medical conditions of the individual.

### CROSS REFERENCE TO RELATED APPLICATIONS

This Application claims priority of U.S. Provisional Application No. 61/578,091, filed on Dec. 20, 2011, the entirety of which is incorporated by reference herein.

### BACKGROUND OF THE INVENTION

1. Field of the Invention

The invention relates to an apparatus for providing medical diagnosis, and more particularly, to an apparatus for providing voice-assisted medical diagnosis.

2. Description of the Related Art

Nowadays, people receive medical diagnosis and health information by going to a hospital. For patients having chronic diseases, it is important to track their long-term health conditions. Therefore, patients have to go to hospitals periodically, costing the patients a lot of time.

In another aspect, many medical diagnosis technologies use various signals, such as blood pressure, electrocardiogram and brain waves, to diagnose for diseases. However, a voice signal of an individual may be used to assist diagnosis of some diseases, especially chronic diseases. For example, a decline in verbal ability may be early signs of certain diseases, such as dementia and Parkinson's disease. Nevertheless, changes in verbal ability and differences of changes in verbal ability between different diseases or different stages of a disease might be difficult to be recognized by a human. For example, in an early stage of Parkinson's disease, a patient is usually not aware of small declines in their verbal abilities. Therefore, such a patient may not realize early signs of a disease and miss out on early detection and treatments.

### BRIEF SUMMARY OF THE INVENTION

In view of this, the invention provides an apparatus to diagnose certain diseases or/and to track and analyze health conditions of an individual by matching a voice signal of the individual with voice models.

One embodiment of the invention provides an apparatus for use in voice assisted medical diagnosis, comprising: a database, storing a voice model associated with an individual; an input unit, receiving a voice signal from the individual; a voice matching unit, matching the voice signal with the voice model; and a diagnosis unit, diagnosing whether or not the individual suffers from one or a multiple of predetermined diseases according to a matching result from the voice matching unit.

The apparatus further comprises a voice training module, generating the voice model from the individual's voice.

The apparatus further comprises a speech recognition unit, analyzing the individual's voice response to a plurality of predetermined questions to determine one or a multiple of medical conditions of the individual, wherein the diagnosis unit diagnoses whether or not the individual suffers from one or a multiple of the predetermined diseases according to the matching result from the voice matching unit and the one or multiple of medical conditions of the individual.

The apparatus further comprises an alarm unit, giving a warning to the individual if the matching result from the voice matching unit reaches or passes a predetermined threshold.

Another embodiment of the invention provides an apparatus for use in voice assisted medical diagnosis, including: a database, storing a plurality of voice models associated with predetermined diseases; an input unit, receiving a voice signal from an individual; a voice matching unit, matching the voice signal with the plurality of voice models; and a diagnosis unit, diagnosing whether or not the individual suffers from one or a multiple of the predetermined diseases according to a matching result from the voice matching unit.

The apparatus further comprises a speech recognition unit, analyzing the individual's voice response to a plurality of predetermined questions to determine one or a multiple of medical conditions of the individual, wherein the diagnosis unit diagnoses whether or not the individual suffers from one or a multiple of the predetermined diseases according to the matching result from the voice matching unit and the one or multiple of medical conditions of the individual.

Another embodiment of the invention provides a method for diagnosis with assistance of voice, comprising: receiving a voice signal from an individual; matching the voice signal with a voice model associated with the individual and generating a matching result; and diagnosing whether or not the individual suffers from one or a multiple of predetermined diseases according to the matching result.

The method further comprises a step of generating the voice model from the individual's voice.

The method further comprises steps of analyzing the individual's voice response to a plurality of predetermined questions to determine one or a multiple of medical conditions of the individual; and diagnosing whether or not the individual suffers from one or a multiple of the predetermined diseases according to the matching result and the one or multiple of medical conditions of the individual.

The method further comprises a step of giving a warning to the individual if the matching result from the voice matching unit reaches or passes a predetermined threshold.

Still another embodiment of the invention provides a method for diagnosis with assistance of voice, comprising: receiving a voice signal from an individual; matching the voice signal with a plurality of voice models associated with predetermined diseases and generating a matching result; and diagnosing whether or not the individual suffers from one or a multiple of the predetermined diseases according to the matching result.

The method further comprises steps of analyzing the individual's voice response to a plurality of predetermined questions to determine one or a multiple of medical conditions of the individual; and diagnosing whether or not the individual suffers from one or a multiple of the predetermined diseases according to the matching result and the one or multiple of medical conditions of the individual.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 is a block diagram of one embodiment of an apparatus for use in voice assisted medical diagnosis;

FIG. 2 is a block diagram of another embodiment of an apparatus for use in voice assisted medical diagnosis.

### DETAILED DESCRIPTION OF THE INVENTION

The following description is of the best-contemplated mode of carrying out the invention. This description is made for the purpose of illustrating the general principles of the invention and should not be taken in a limiting sense.

As described in the description of the related art, a voice signal of an individual may be used to assist in the diagnoses of some diseases. To construct an apparatus for use in voice assisted medical diagnosis, the basic idea of the invention is to build voice models. In one embodiment, a voice model includes of some voice or/and phonetic characteristics, such as pitch, tones, tempo, articulation, volume, sound waves, clarity, intervals, fluency, syllable, stress, vowel, consonant etc. These voice or/and phonetic characteristics may be determined by linguistics parameters, such as phonology or/and phonetics. For example, a voice signal fluency may be determined by whether intervals are placed correctly or/and the number of intervals. And the fluency may also be determined according to phone time ratio, articulation, silence pause count, total duration of pauses and mean length of pauses.

One embodiment of the invention builds a plurality of voice models associated with different diseases, respectively. For example, the embodiment of the invention builds a voice model associated with dementia and a voice model associated with Parkinson's disease. To be noted, a disease may be associated with more than one voice model. By matching a voice signal of an individual with the plurality of voice models, the embodiment of the invention may determine whether the voice signal is similar to one or a multiple of the plurality of voice models. If the voice signal highly matches one of the plurality of voice models, the embodiment of the invention diagnoses that the individual suffers from a disease associated with the one of the plurality of voice models. For example, for patients suffering from dementia, it is hard for them to repeat some vowel patterns correctly, for example, “bee-bah-boh”. A patient suffering from dementia might say “bee-boh-boh” or “bee-bee-bee” when the patient is asked to repeat “bee-bah-boh” four times. Therefore, when an individual is asked to repeat “bee-bah-boh” four times, the voice signal of the individual is recorded and matched with some voice models associated with verbal performances of “bee-bah-boh” of dementia to determine whether the individual suffers from dementia. Furthermore, when matching the voice signal with the voice models, lengths of intervals between “bee-bah-boh” repeated for four times may also be considered. Also, voice models associated with diseases may be built in different scripts. For example, instead of or in addition to “bee-bah-boh”, “bee-key-gee” is also a test script for dementia.

The plurality of voice models may be built in different sets corresponding to different genders, different ages or/and different languages. Therefore, the voice signal of the individual is matched with a set of voice models corresponding to the gender, the age or/and language used by the individual.

Another embodiment, by matching a voice signal with voice models, not only diagnoses whether an individual suffers from a disease but also determines which stage of a disease an individual is suffering from. For example, a set of voice models corresponding to a disease includes a number of voice models, wherein each of the number of voice models is associated with one stage of the disease.

Another embodiment of the invention includes a voice signal of an individual with a voice model of the individual obtained from a period of time ago to trace a change in the health condition of the individual. For example, if a difference between the voice signal and a voice model of the individual obtained one month ago is larger than a predetermined threshold, the change in health condition of the individual is determined to be severe, and the health condition of the individual is determined to possibly worsen.

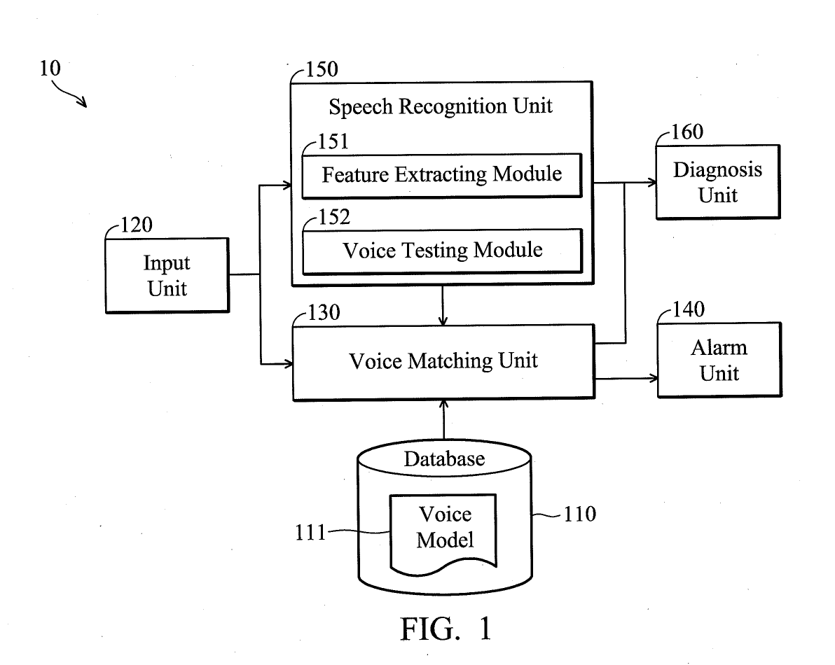

FIG. 1 is a block diagram of one embodiment of an apparatus 10 for use in voice assisted medical diagnosis. As shown in FIG. 1, the apparatus 10 includes a database 110, an input unit 120, a voice matching unit 130, an alarm unit 140, a speech recognition unit 150 and a diagnosis unit 160.

A voice model 111 associated with an individual is stored in the database 110. The voice model 111 may be a voice model obtained from the individual from a period of time ago. The input unit 120 receives a voice signal from the individual. The voice matching unit 130 matches the voice signal with the voice model 111. The diagnosis unit 160 diagnoses whether or not the individual suffers from one or a multiple of predetermined diseases according to a matching result from the voice matching unit 130.

The alarm unit 140 gives a warning to the individual if the matching result from the voice matching unit 130 reaches or passes a predetermined threshold. For example, if the difference between the voice signal and the voice model 111 is big, the health condition of the individual is determined to possibly worsen.

In one example, the voice signal of the individual is recorded when the individual reads out loud one or a multiple of predetermined scripts. The predetermined script is provided to the individual by an output unit (not shown), such as a display or an audio player.

The speech recognition unit 150 analyzes the individual's voice response to a plurality of predetermined questions to determine one or a multiple of medical conditions of the individual. The plurality of predetermined questions may be provided to the individual by the output unit. The plurality of predetermined questions may be designed to get physical or/and mental information of the individual. For example, the plurality of predetermined questions is displayed on a screen. The input unit 120 receives the individual's voice response, such as answers to the plurality of predetermined questions. Then the speech recognition unit 150 uses speech recognition to retrieve keywords of the individual's voice response and uses the keywords to determine one or a multiple of medical conditions of the individual according to a statistical analysis between keywords and medical conditions. In another embodiment, a hand writing panel or a keyboard may be used to input the individual's answers, and a processing unit uses text recognition to retrieve keywords from the answers and determine one or a multiple of medical conditions of the individual according to the keywords. When determining one or a multiple of medical conditions of the individual, some parameters may also be considered, such as typing strength or the response time to the plurality of predetermined questions.

The diagnosis unit 160 utilizes a statistical analyzing method to diagnose whether or not the individual suffers from one or a multiple of predetermined diseases according to the matching result from the voice matching unit 130 and the one or multiple of medical conditions of the individual determined by the speech recognition unit 150. Therefore, the apparatus 10 diagnoses whether or not the individual suffers from one or a multiple of the predetermined diseases according both changes in voice of the individual and medical conditions of the individual.

In another example, the apparatus 10 may further include a voice training module (not shown). The voice training module generates the voice model from the individual.

In another example, the apparatus 10 may further include a voice processing unit (not shown). The processing unit retrieves voice or/and phonetic characteristics of the voice signal and provides the characteristics to the voice matching unit 130. Then the voice matching unit 130 utilizes the characteristics to match the voice signal with the voice model 111. For instance, the voice matching unit determines a score according to a matching in the characteristics between the voice signal and the voice model 111, and the score represents a difference between the voice signal and the voice model 111. The alarm unit 140 gives a warning to the individual if the score is larger than a predetermined value.

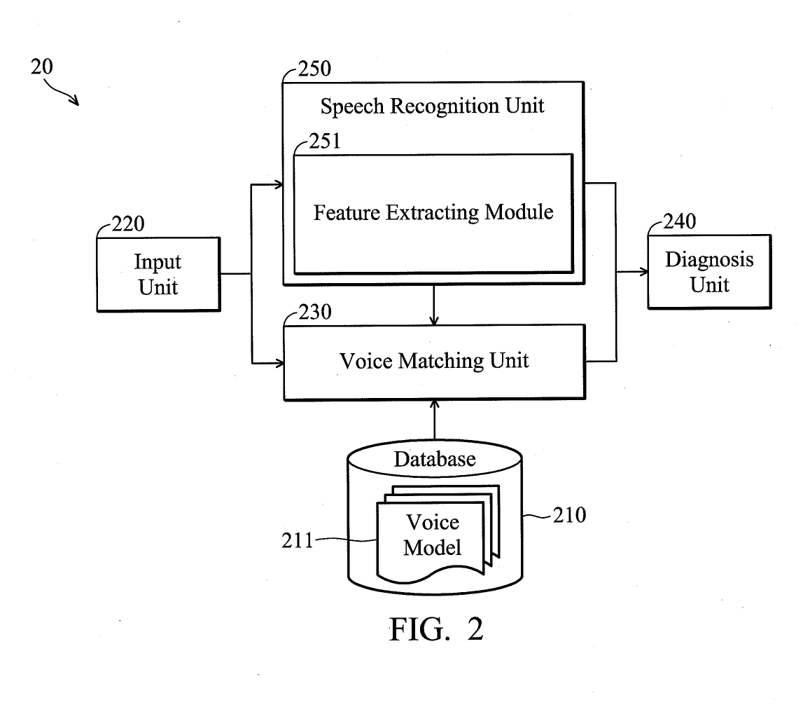

FIG. 2 is a block diagram of another embodiment of an apparatus 20 for use in voice assisted medical diagnosis. As shown in FIG. 2, the apparatus 20 includes a database 210, an input unit 220, a voice matching unit 230, a diagnosis unit 240 and a speech recognition unit 250. The input unit 220 receives a voice signal from the individual.

A plurality of voice models 211 associated with predetermined diseases is stored in the database 210. The plurality of voice models 211 may be constructed according to a plurality of predetermined scripts that represents at least one significant characteristic relating to a predetermined disease. The voice matching unit 230 matches the voice signal with the plurality of voice models 211. The diagnosis unit 240 diagnoses whether or not the individual suffers from one or a multiple of the predetermined diseases according to a matching result from the voice matching unit 230.

In one example, at least one predetermined script is provided to the individual by an output unit (not shown), such as a display or an audio player. The input unit 220 records the voice signal of the individual when the individual reads out loud the at least one predetermined script provided by the output unit.

In another example, the diagnosis unit 240 utilizes a statistical analyzing method to diagnose whether or not the individual suffers from one or a multiple of the predetermined diseases according to not only the matching result from the voice matching unit 230 but also one or a multiple of medical conditions of the individual determined by the speech recognition unit 250. Similar to the speech recognition unit 150 in FIG. 1, the speech recognition unit 250 analyzes the individual's voice response to a plurality of predetermined questions to determine the one or multiple of medical conditions of the individual.

The apparatus 20 may further include a voice training module (not shown). The voice training module generates the voice model from the individual.

The apparatus 20 may further include a voice processing unit (not shown). The processor retrieves voice or/and phonetic characteristics of the voice signal and provides the characteristics to the voice matching unit 230. Then the voice matching unit 230 utilizes the characteristics to match the voice signal with some of the plurality of voice models 211 that is related to the predetermined script. If the voice signal matches one or a multiple of the plurality of voice models 211, the diagnosis unit 240 diagnoses that the individual suffers from one or a multiple of the predetermined diseases that is associated with the matched voice models.

In another example, the database 110 and 210 may also store an anamnesis file of the individual. The diagnosis unit 160 and 240 may use the anamnesis file as a reference to assist in diagnosing the individual.

As described above, the present disclosure provides an apparatus for use in voice assisted medical diagnosis to diagnose some disease, such as dementia and any other disease having changes in voice or/and phonetic characteristics. The apparatus of the present disclosure may also track a patient's condition and give a warning to the patient if conditions worsen.

The input unit 120 and 220 may be a microphone or a mobile phone. The database 110 and 210 may be a storage device such as a hard disk device. The voice matching unit 130 and 230, the alarm unit 140, the diagnosis unit 160 and 240 and the speech recognition unit 150 and 250 may be processors that are able to implement the functions as described above, respectively. For example, the voice matching unit 130 and 230 and the speech recognition unit 150 and 250 may be audio signal processors.

In another embodiment, the database 110 and 210, the voice matching unit 130 and 230, the diagnosis unit 160 and 240 and the speech recognition unit 150 and 250 may be implemented entirely in the form of a server computer configured with computer executable instructions for causing the functions thereof to be performed. The input unit 120 and 220 may be a communication device that may receive voice signals. The server computer is connected to a communications network. The communication device is also connected to the network and in data communication with the server computer via the network. For example, the predetermined scripts and the predetermined questions may be displayed on a screen of a mobile phone, and the voice signal of the individual is received by the receiver of the mobile phone and is sent via a network to a remote server computer to diagnose whether the individual suffers from one or a multiple of predetermined diseases or/and to track the individual's condition. If a matching result from the voice matching unit of the remote server computer reaches or passes a predetermined threshold, the remote server computer sends a warning signal to the mobile phone and a warning message may be displayed on the screen or played by a speaker of the mobile phone to inform the individual of the warning message. If a diagnosis unit of the remote server computer diagnoses that the individual suffers from a disease, the remote server computer sends a diagnosis and medical advice via the network to the mobile phone.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An apparatus for use in voice assisted medical diagnosis, comprising:
a database, storing a voice model associated with an individual;
an input unit, receiving a voice signal from the individual;
a voice matching unit, matching the voice signal with the voice model; and
a diagnosis unit, diagnosing whether or not the individual suffers from one or a multiple of predetermined diseases according to a matching result from the voice matching unit.

2. The apparatus as claimed in claim 1, further comprising:
a voice training module, generating the voice model from the individual's voice.

3. The apparatus as claimed in claim 1, further comprising:
a speech recognition unit, analyzing the individual's voice response to a plurality of predetermined questions to determine one or a multiple of medical conditions of the individual,
wherein the diagnosis unit, diagnoses whether or not the individual suffers from one or a multiple of the predetermined diseases according to the result from the voice matching unit and the one or multiple of medical conditions of the individual.

4. The apparatus as claimed in claim 1, further comprising:
an alarm unit, giving a warning to the individual if the matching result from the voice matching unit reaches or passes a predetermined threshold.

5. An apparatus for use in voice assisted medical diagnosis, comprising:
a database, storing a plurality of voice models associated with predetermined diseases;
an input unit, receiving a voice signal from an individual;
a voice matching unit, matching the voice signal with the plurality of voice models; and
a diagnosis unit, diagnosing whether or not the individual suffers from one or a multiple of the predetermined diseases according to a matching result from the voice matching unit.

6. The apparatus as claimed in claim 5, further comprising:
a speech recognition unit, analyzing the individual's voice response to a plurality of predetermined questions to determine one or a multiple of medical conditions of the individual,
wherein the diagnosis unit diagnoses whether or not the individual suffers from one or a multiple of the predetermined diseases according to the result from the voice matching unit and the one or multiple of medical conditions of the individual.

7. A method for diagnosis with assistance of voice, comprising:
receiving a voice signal from an individual;
matching the voice signal with a voice model associated with the individual and generating a matching result; and
diagnosing whether or not the individual suffers from one or a multiple of predetermined diseases according to the matching result.

8. The method as claimed in claim 7, further comprising:
generating the voice model from the individual's voice.

9. The method as claimed in claim 7, further comprising:
analyzing the individual's voice response to a plurality of predetermined questions to determine one or a multiple of medical conditions of the individual; and
diagnosing whether or not the individual suffers from one or a multiple of the predetermined diseases according to the matching result and the one or multiple of medical conditions of the individual.

10. The method as claimed in claim 7, further comprising:
giving a warning to the individual if the matching result from the voice matching unit reaches or passes a predetermined threshold.

11. A method for diagnosis with assistance of voice, comprising:
receiving a voice signal from an individual;
matching the voice signal with a plurality of voice models associated with predetermined diseases and generating a matching result; and
diagnosing whether or not the individual suffers from one or a multiple of the predetermined diseases according to the matching result.

12. The method as claimed in claim 11, further comprising:
analyzing the individual's voice response to a plurality of predetermined questions to determine one or a multiple of medical conditions of the individual; and
diagnosing whether or not the individual suffers from one or a multiple of the predetermined diseases according to the matching result and the one or multiple of medical conditions of the individual.
